(19) United States
(12) Patent Application Publication
(10) Pub. No.: US 2005/0202096 A1
(43) Pub. Date: Sep. 15, 2005

(54) Microcapsules for encapsulation of bioactive substances

(76) Inventors: Jun Li, Singapore (SG); Chai Hoon Quek, Singapore (SG); Leong Ming Gan, Singapore (SG); Hanry Yu, Irvine, CA (US); Kam W. Leong, Ellicott City, MD (US)

Correspondence Address: Madeline I. Johnston;King & Spalding, LLP, 191 Peachtree Street, Atlanta, GA, 30303-1763, US

(21) Appl. No.: 10/796,902

(22) Filed: Mar. 9, 2004

(51) Int. Cl. A61K 9/16; A61K 9/50

(52) U.S. Cl. 424/490; 424/93.7

(57) ABSTRACT

The invention relates to photo-crosslinkable microcapsules with enhanced properties for encapsulation of bioactive substances. The present invention also relates to a microencapsulation composition and to a method of manufacturing microcapsules. The microcapsule comprise bioactive substances that are encapsulated by a selectively permeable polymeric membrane. The selectively permeable membrane is impermeable to immune system components associated with immune rejection in an animal. A monomer of the selectively permeable polymeric membrane includes a photosensitive cross-linking agent having a cinnamoyl moiety.

### TECHNICAL FIELD

The present invention generally relates to microcapsules for encapsulation of bioactive substances such as living cells and therapeutic agents. The present invention also relates to a composition for the production of microcapsules and to a method of manufacturing microcapsules.

### BACKGROUND

Microencapsulation systems provide the separation of bioactive substances such as living cells from the immune system of the body. Microencapsulation systems involve the use of a synthetic microcapsule having a selectively permeable membrane for encapsulating the bioactive substances. The selective membrane allows the free exchange of nutrients, oxygen and biotherapeutic substances between the blood or plasma and the encapsulated bioactive substances, whereas high molecular weight substances such as immunocytes, antibodies and other transplant rejection effector mechanisms are excluded. The microcapsules may also modulate the bi-directional diffusion of antigens, cytokines and other immunological moieties based on the chemical characteristics of the membrane and matrix support.

Microencapsulation systems provide a solution to the problem of donor organ supply, not only by potentially allowing the transplantation of allogeneic cells and tissues without immunosuppression, but also by permitting use of xenogenic cells.

Early microencapsulation systems were used to encapsulate proteins within microcapsules having a semipermeable polymer membrane. Microencapsulation systems have also been used for the immobilization of a variety of biologically active species such as enzymes and living cells, which have been used in the development of bioreactors, biosensors, and hybrid bioartificial organs.

One known microencapsulation system has involved the encapsulation of islets using a complexation of polyanionic alginate with polycationic poly(L-lysine) (PLL), and implantation of the microcapsules into rats with streptozotocin-induced diabetes [Lim F., et al., Microencapsulated islets as bioartificial endocrine pancreas, Science 1980; 210: 908-910]. However, the PLL polycation is costly and has limited mechanical properties and biocompatibility.

Another microencapsulation system that utilizes biocompatible microcapsules for use in bioartificial liver assist devices (BALD) is to encapsulate primary rat hepatocytes in two layers of a polymeric membrane; an inner layer and an outer layer. The inner layer consists of a cationic biopolymer substrate that is soluble at physiological pH and body temperature. The outer layer is a synthetic anionic polyelectrolyte copolymer. Hepatocyte cells are suspended in a solution of modified collagen and added to the solution of the inner layer to form microcapsules through complex coacervation reaction. The membrane of the capsules is permeable to nutrients that are required to maintain the metabolic functions of the cells. Products secreted by the cells will diffuse out of the capsules, which provides immunological protection to the cells by restraining the migration of antibodies and cells across the membrane. However, the disadvantage with these known type of microcapsules is that they have weak mechanical properties. Without being bound by theory, it is thought that these weak mechanical properties occur due to one or more of the following reasons:
(1) The microcapsules are highly hydrated;
(2) The collagen layer is subject to degradation by extracellular enzymes; and
(3) The interaction between the polymeric layers is through the ionic bonding instead of the stronger covalent bonding.

Efforts have been made to improve the mechanical strength of the microcapsules by a two-step encapsulation process to form microcapsules with four separate layers comprising two ter-copolymer shells spaced by two layers of modified collagen or to create a macro-porous exoskeleton for microcapsules over the polymer-collagen two-layer shells with alumina and chitosan. However, introducing the additional layers requires precise control of the experimental conditions so as not to rupture the initially formed microcapsules. The required precise control increases costs and renders scale-up production of such microcapsules very difficult to achieve.

A method that has been investigated to increase the strength of microcapsules involved the formation of covalent bonds between alginate and polycations by the use of a photosensitive agent. [Lu, M. Z. et al., Cell Encapsulation with Alginate and α-Phenoxycinnamylidene-Acetylated Poly(Allylamine) Biotechnology and Bioengineering, Vol 70, No. 5, December 2000: 479-483, and Lu, M. Z. et al, A novel cell encapsulation method using photo-sensitive poly(allylamine alpha-cyanocinnamylideneacetate) Journal of Microencapsulation, 2000, Vol 17, No. 2: 245-251]. The method involved two kinds of photosensitive poly(allyamine) synthesized with 5% and 10% of amino groups modified by α-phenoxycinnamylideneacetyl chloride. The photosensitive polymers were irradiated with UV light in the range between 300-325 nm to form cross-links between the monomer groups. The maximum wavelength at which the photosensitive monomers formed cross-linked groups was 325 nm. Although the strength of the microcapsules prepared by this method did increase somewhat, damage of the encapsulated cells within the microcapsules occurred when the monomers were subjected to radiation at wavelengths in the range between 300-325 nm.

There is a need to provide microcapsules for encapsulation of bioactive substances that overcome or at least ameliorate one or more of the disadvantages associated with the prior art above.

There is a need to provide photo-crosslinkable microcapsules for encapsulation of living cells and biomaterials having good chemical and mechanical stability.

There is a need to provide photo-crosslinkable microcapsules for encapsulation of living cells with minimal, reduced or no damage to encapsulated cells upon exposure to UV light.

### SUMMARY OF INVENTION

According to a first aspect of the invention, there is provided a microcapsule comprising:
a permeable polymer membrane comprising a plurality of cinnamoyl groups, the cinnamoyl groups being capable of forming cross-links upon exposure to radiation; and
a bioactive substance encapsulated by the permeable membrane.

According to a second aspect of the invention, there is provided a microcapsule comprising:
a permeable polymer membrane comprising at least one crosslinking group derived from at least two cinnamoyl groups; and
a bioactive substance encapsulated by the permeable polymer membrane.

According to a third aspect of the invention, there is provided a microcapsule comprising:
a permeable polymer membrane comprising:
an inner polymer layer comprising a first polymer having a first electrical charge;
an outer polymer layer comprising a second polymer having a second electrical charge opposite to the first electrical charge, the second electrical charge being sufficient to form a complex with the first polymer of the inner polymer layer;
a plurality of monomer groups comprising cinnamoyl groups incorporated into a backbone of the first polymer or the second polymer, the cinnamoyl groups being capable of forming cross-links when exposed to light at a wavelength in the range from 340 nm to 700 nm; and
a bioactive substance encapsulated by the permeable membrane.

According to a fourth aspect of the invention, there is provided a method of preparing a microcapsule, comprising the steps of:
(a) providing a first solution of a first polymer;
(b) providing a second solution of a second polymer;
(c) providing a bioactive substance in the first or the second polymers; and
(d) introducing the first solution, the second solution and the bioactive substance to form a permeable polymer membrane at least partially surrounding the bioactive substance;
wherein at least one of the first polymer and the second polymer has a plurality of cinnamoyl groups capable of forming cross-links upon exposure to radiation.

The method may comprise the further step of:
(e) exposing the permeable polymer membrane to radiation so that the cinnamoyl groups form cross-links within the permeable polymer membrane.

According to a fifth aspect of the invention, there is provided a pharmaceutical composition comprising a pharmacologically effective plurality of the microcapsules of the first aspect or the second aspect, together with a pharmacologically acceptable carrier.

According to a sixth aspect of the invention, there is provided use of one or more microcapsules as defined in the first aspect or the second aspect, in a liver assist device.

According to a seventh aspect of the invention, there is provided use of one or more microcapsules as defined in the first aspect or the second aspect, as a stem cell scaffold material.

According to an eighth aspect of the invention, there is provided use of one or more microcapsules as defined in the first aspect or the second aspect, in the controlled delivery of therapeutic agents.

### DEFINITIONS

The following words and terms used herein shall have the meaning indicated:

The term ‘bioactive substance’ is to be interpreted broadly to include cells, such as living cells or genetically modified cells or any natural or synthetic material that causes a biological response in living tissue.

The word “biopolymer” and grammatical variations thereof are to be interpreted broadly to include any polymer that is biologically compatible by not producing a toxic, injurious, or immunological response in living tissue.

### Disclosure of Embodiments

The disclosed embodiments relate to microcapsules for encapsulation of bioactive substances and more particularly photo-crosslinkable microcapsules with enhanced properties, such as mechanical strength and chemical stability.

The disclosed embodiments describe a novel microcapsule for encapsulating bioactive substances such as hepatocyte cells. The microcapsule comprises a permeable polymer membrane comprising a plurality of cinnamoyl groups. The cinnamoyl groups are capable of forming cross-links upon exposure to radiation. A bioactive substance is encapsulated by the permeable membrane.

The bioactive substance may be a living cell and may be provided in a medium having a composition to maintain cell function. The bioactive substance is enclosed by the permeable membrane which may be selectively permeable. The selectively permeable membrane may be impermeable to one or more immune system components associated with immune rejection in an animal and may be permeable to one or more materials necessary to sustain the normal metabolic functions of the cells and to one or more products released by the cells.

The embodiments also describes a novel composition for use in preparing microcapsules for encapsulating bioactive substances and a novel method of preparing such microcapsules.

The disclosed embodiments provide photo-crosslinkable microcapsules for encapsulation of living cells, wherein the crosslinks are formed by causing minimal damage to the living cells upon exposure to UV light.

In one embodiment, there is provided a permeable membrane comprising a first polymer, a second polymer and a plurality of cinnamoyl groups in at least one of the polymers. A bioactive substance is encapsulated by the permeable membrane. The cinnamoyl groups act as photosensitive cross-linking agents that may be exposed to ultraviolet light or visible light at wavelengths that cause minimal, or reduced or no damage to cells located in the core of the microcapsules. The ultraviolet light advantageously causes cross-linking to occur between the monomers of the polymeric membrane to increase mechanical strength and chemical stability.

The first polymer and second polymer may be capable of adhering to each other. The first and second polymers may be oppositely charged biopolymers that may form respective inner and outer polymer layers. The cinnamoyl groups may be provided in the outer polymer layer or alternatively in the inner polymer layer.

There may be provided a monomer group having a cinnamoyl group incorporated into a polymer backbone of the first polymer or the second polymer.

The Bioactive Substance

The bioactive substance may be cells or non-cellular material that causes a biological response in living tissue.

The cells that may be encapsulated by the permeable membrane include living cells and genetically engineered cells. The cells may be selected from the group consisting of: hepatocyte cells, hematopoietic cells, epithelial cells, secretory cells, ciliated cells, contractile cells, sensory cells and neuronal cells and one or more combinations thereof.

The hematopoietic cells may be selected from the group consisting of granulocytes, lymphocytes, macrophages/monocytes, red blood cells and one or more combinations thereof.

The epithelial cells may be selected from the group consisting of keratinizing epithelial cells, epithelial cells specialized for exocrine secretion, epithelial absorptive cells and one or more combinations thereof.

The secretory cells may be selected from the group consisting of Leydig cells, cells of thyroid gland, adrenal gland, islets of Langerhans and one or more combinations thereof.

The ciliated cells may be selected from the group consisting of respiratory tract cells, oviduct and endometrium of uterus cells, rete testis and ductulus efferens cells, ependymal cells lining brain cavities and one or more combinations thereof.

The contractile cells may be selected from the group consisting of skeletal muscle cells, heart muscle cells, smooth muscle cells and one or more combinations thereof.

The sensory cells may be selected from the group consisting of photoreceptor cells, olfactory neuron cells, hair cells of organ of Corti, taste bud cells, and one or more combinations thereof.

The neuronal cells may be selected from the group consisting of neuron cells, glial cells and one or more combinations thereof.

The bioactive substance may be selected from the group consisting of: therapeutic compounds, neurologics, vitamins, vitamin derivatives, growth factors, glucocorticosteroids, steroids, antibiotics, anti-bacterial compounds including bacteriocidal and bacteriostatic compounds, anti-viral compounds, anti-fungal compounds, anti-parasitic compounds, tumoricidal compounds, tumoristatic compounds, toxins, enzymes, enzyme inhibitors proteins, peptides, minerals, neurotransmitters, lipoproteins, glycoproteins, immunomodulators, immunoglobulins and corresponding fragments, dyes, radiolabels, radiopaque compounds, fluorescent compounds, fatty acid derivatives, polysaccharides, cell receptor binding molecules, anti-inflammatories, anti-glaucomic compounds, mydriatic compounds, anesthetics, nucleic acids, polynucleotides and combinations of one ore more thereof.

The Permeable Membrane

The permeable membrane may be a selectively permeable membrane. The selectively permeable membrane may be impermeable to bacteria, lymphocytes, large proteins, and other entities of the type responsible for immunological reactions that result in rejection of cells from the host's immune system.

The selectively permeable membrane may be permeable to nutrients, ions, oxygen, and other materials necessary to sustain the normal metabolic functions of the cell, as well as to products released by the cell, such as insulin released in response to glucose, urea, bilirubin and bile salts from hepatic cells.

The selectivity of the selectively permeable membrane may be modified according to the molecular weight of the polymers comprising the membrane.

The selectivity of the selectively permeable membrane may be controlled by providing at least one hydrophobic group and at least one hydrophilic group in the outer polymer layer. The hydrophilic group may be provided adjacent to the inner polymer layer and the outer polymer layer may at least partially surround the inner polymer layer.

The selectively permeable membrane may be formed by complex coacervation of two oppositely charged polymer layers if the polymers have sufficient charge density to cohere.

The selectively permeable membrane may have comprise oppositely charged outer and inner polymer layers. The outer or inner polymer layer, or both, having a photosensitive cross-linking agent.

The Permeable Membrane: The Cinnamoyl Groups

One of the polymer layers is provided with a plurality of cinnamoyl groups that act as photosensitive cross-linking agents. The cinnamoyl groups cause cross-linking within the polymer layers upon exposure to radiation.

A monomer group may be provided with the cinnamoyl group, which may be incorporated into the polymer backbone. The monomer with the cinnamoyl group may be a monomer that undergoes a [2+2] photocycloaddition reaction. The monomer with the cinnamoyl group may be any cinnamoyl derivative that causes cross-linking upon exposure to radiation within the polymer layers. The monomer with the cinnamoyl group may be a monomer selected from the group consisting of: 4-(4-Methoxycinnamoyl)phenyl methacrylate; 3,4-dimethoxycinnamoyloxyethyl methacrylate, 3,4,5-trimethoxycinnamoyloxyethyl methacrylate, cinnamoyloxyethyl methacrylate and combinations thereof.

The Permeable Membrane: The Inner Polymer Layer

The inner polymer layer may be comprised of either a cationic or anionic biopolymer that is water soluble and may typically have a weight of more than about 200,000. The inner polymer layer may be moderately hydrophobic.

The polymer of the inner polymer layer may be a naturally-occurring biopolymer, a modified biopolymer or a synthetic biopolymer. The naturally occurring biopolymer may be collagen, which may be modified by raising its pKI to a sufficient charge such that it forms a cationic polymer at physiological pH which is able to form a complex with the outer polymer layer. In one embodiment, the collagen is modified to have a pKI of at least 9.

Alternatively, the collagen may be modified to form an anionic polymer and thereby form a complex with a cationic outer polymer layer by converting the primary amino groups to tertiary amino groups or by esterification.

In one embodiment, “esterified collagen” is used as a biopolymer for use in the inner polymer layer. Esterified collagen involves the collagen undergoing a reaction to form tertiary amine groups. The collagen may be reacted with a wide variety of aliphatic reactants containing as many as about 18 carbon atoms in their chain and include alcohols, primary amines and alcohol amines. Reactants having about 8 carbon atoms or less and for certain purposes, reactants having only 2 or 3 carbon atoms may be used. The alcohols that may be used include methanol, ethanol, butanol and higher alcohols. The primary amines that may be used include methylamine, ethylamine and higher amines. Reactants with both alcohol and amine groups, such as ethanolamine, may also be used.

Alternative cationic biopolymers that may be used for the inner polymer layer include high molecular weight proteins such as fibrin, polylysine and the like. Cationic biopolymers having a pKI of at least about 9 may be used and more usefully at least about 10.

In other embodiments, the inner polymer layer may be comprised of anionic biopolymers such as hyaluronic acid (HA) and “modified HA” (HA partially esterified or reacted with a primary amine to render it less water soluble). Advantageously, modified HA forms a stronger complex with a polycationic outer layer than HA itself. Suitably, such anionic biopolymers suitable for use in the inner polymer layer have a charge density of at least about 50%.

The Permeable Membrane: The Outer Polymer Layer

The outer polymer layer may comprise a biopolymer such as a biocompatible synthetic polyelectrolyte that has an opposite charge to the biopolymer of the inner polymer layer. The biopolymer of the outer polymer layer may have a molecular weight of at least about 200,000.

In an embodiment where the biopolymer of the inner polymer layer is polycationic, such as modified collagen, the synthetic polyelectrolyte used in the outer polymer layer is polyanionic.

In another embodiment where the biopolymer of the inner polymer layer is polyanionic, such as HA and modified HA, the synthetic polyelectrolyte used in the outer polymer layer is polycationic.

In one embodiment, a class of biocompatible synthetic polyelectrolytes that may be used in the outer polymer layer are acrylate polymers.

Cationic synthetic polymers may be selected from the group consisting of acrylate polymers, copolymers and terpolymers such as poly(acrylic acid), poly(methacrylic acid) poly(methacrylate), poly(methyl methacrylate) and acrylate copolymers and terpolymers of acrylic acid, methacrylic acid, methacrylates, methyl methacrylates, hydroxyethyl methacrylic such as 2-hydroxyethyl methacrylate, hydroxypropylacrylate and one or more combinations thereof.

Anionic synthetic polymers may be selected from the group consisting of poly(dimethylaminoethyl methacrylate) (“DMAEMA”) and copolymers and terpolymers of dimethylaminoethyl methacrylate with 2-hydroxyethyl methacrylate and/or hydroxypropylacrylate and methacrylate and/or methyl methacrylate; copolymers or terpolymers of acrylic acid and/or methacrylic acid with 2-hydroxyethyl methacrylic and/or hydroxypropylacrylate and methacrylate and/or methyl methacrylate.

In one embodiment, the monomer having the cinnamoyl groups is included with one or more monomers of the outer polymer layer. An exemplary schema for synthesis of a tetra-copolymer system that includes a monomer having a photosensitive cross-linking agent is shown in FIG. 2 and will be described in more detail below. FIG. 2 shows synthesis of a HEMA-MMA-MAA-MeOCPMA tetra-copolymer system in which 2-hydroxyethyl methacrylate (HEMA), methyl methacrylate (MMA), methacylic acid (MAA) and 4-(4-methoxycinnamoyl)phenyl methacrylate (MeOCPMA). The MeOCPMA monomer contains a cinnamoyl moiety that in the schema of FIG. 2 is incorporated in the backbone of the tetra-copolymer. The description of the preparation of the tetra-copolymer follows further below.

Process for Making Microcapsules

The embodiments discloses a novel process of preparing microcapsules for the encapsulation of bioactive substances. The process involves providing a first polymer solution containing monomers of the inner polymer layer described above and having a first electrolytic charge. The first polymer solution may contain a suspension of the bioactive substance as described above.

The method further includes providing a second polymer solution containing monomers of the outer polymer layer described above and having a second electrolytic charge opposite to the polymers of the first polymer solution.

One of the monomers of the first or second polymer solutions includes the cinnamoyl groups as described above.

The electrolytic charge between the first and second polymer solutions may be such that a complex is formed between the first polymer solution and the second polymer solution to form a permeable membrane. The permeable membrane surrounding the bioactive substance.

Once a complex has been formed between the first and second polymer solutions, the complex may be exposed to radiation to form cross-links between the monomers of the first or second polymer solution.

In one embodiment the membrane may be formed by the complex coacervation process. The first biopolymer solution may form a complex with a second biopolymer solution comprising one or more of the synthetic polyelectrolytes described above having an opposite charge to the electrolytic charge of the first biopolymer. The first biopolymer solution may form a complex with the second biopolymer solution by being added dropwise to the second biopolymer solution. The charge density of the synthetic polyelectrolytes may be at least about 3%.

The concentration of cells suspended within the first biopolymer solution may be in the range selected from the group consisting of: 1.0×10⁴ cells/mL to 1.0×10⁷ cells/mL; 1.0×10⁵ cells/mL to 5.0×10⁶ cells/mL; 5.0×10⁵ cells/mL to 5.0×10⁶ cells/mL; 1.0×10⁶ cells/mL to 4.0×10⁶ cells/mL; 1.5×10⁶ cells/mL to 3.0×10⁶ cells/mL; and 2.0×10⁶ cells/mL to 3.0×10⁶ cells/mL.

The molar concentration of the monomer having the photosensitive agent within the second biopolymer solution may be in the range selected from the group consisting of: 0.01 mol % to 4 mol %; 0.05 mol % to 3.5 mol %; 0.1 mol % to 3.0 mol %; 0.5 mol % to 2.5 mol %; 1.0 mol % to 2.4 mol %; 1.5 mol % to 2.2 mol %; and 1.8 mol % to 2.1 mol %.

The concentration of biopolymer within the first biopolymer solution may be in the range selected from the group consisting of: 0.1 mg/mL to 5.0 mg/mL; 0.2 mg/mL to 4.5 mg/mL; 0.3 mg/mL to 4.0 mg/mL; 0.4 mg/mL to 3.5 mg/mL; 0.5 mg/mL to 3.0 mg/mL; 0.8 mg/mL to 2.5 mg/mL; 1.0 mg/mL to 2 mg/mL; and 1.2 mg/mL to 1.8 mg/mL.

The concentration of biopolymer within the second biopolymer solution may be in the range selected from the group consisting of: 0.01 wt % to 5.0 wt %; 0.05 wt % to 4.5 wt %; 0.1 wt % to 4.0 wt %; 0.5 wt % to 3.5 wt %; 0.6 wt % to 3.0 wt %; 0.7 wt % to 2.5 wt %; 0.8 wt % to 2.0 wt %; and 0.9 wt % to 1.5 wt %.

The diameter of the formed microcapsules may be in the range selected from the group consisting of: 500 μm to 1500 μm; 600 μm to 1400 μm; 650 μm to 1300 μm; 700 μm to 1200 μm; 750 μm to 1100 μm; 800 μm to 1000 μm; and 850 μm to 950 μm.

The thickness of the outer layer of the formed microcapsules may be in the range selected from the group consisting of: 50 μm to 250 μm; 80 μm to 220 μm; 100 μm to 200 μm; 120 μm to 180 μm; 130 μm to 170 μm; and 140 μm to 160 μm.

Cross-links between the monomers of the first or second polymer layers are formed by subjecting the complex to radiation that may be selected from the group consisting of: ultraviolet light; visible light; gamma rays; and X-ray radiation.

In embodiments where living cells are contained within the biopolymer solution, the radiation may be ultraviolet or visible light in the range selected from the group consisting of: 340 nm to 700 nm; 360 nm to 680 nm; 380 nm to 660 nm; 400 nm to 640 nm; 420 nm to 620 nm; 440 nm to 600 nm; 460 nm to 580 nm.

The microcapsules may be subjected to the radiation for a continuous or discontinuous time period. The time period for subjecting the microcapsules to radiation may be in the range selected from the group consisting of: 1 second to 300 seconds; 5 seconds to 240 seconds; 10 seconds to 200 seconds; 15 seconds to 120 seconds; 20 seconds to 80 seconds; 25 seconds to 70 seconds; 30 seconds to 60 seconds; and 40 seconds to 55 seconds.

In one embodiment, the first biopolymer solution is modified collagen having bioactive substance contained therein. The second biopolymer solution is a tetra-copolymer solution of HEMA, MMA, MAA and MeOCPMA monomers. The cinnamoyl moiety of MeOCPMA can interact in an ionic manner with the cationic modified collagen to form double-layer microcapsules with the modified collagen through ionic interaction. In this embodiment, the outer layer of the double-layer microcapsules is a tetra-copolymer layer of HEMA-MMA-MAA-MeOCPMA and is anionic, while the modified collagen comprises the inner layer and is anionic. The tetra-copolymer can be photo cross-linked with the inner layer of collagen to improve the mechanical strength of the microcapsules. The outer layer may not directly contact the cells which may be incorporated within the inner layer so that the cells are not affected during cross-linking. Furthermore, as the MeOCPMA contains a cinnamoyl moiety, cross-linked groups can be formed at wavelengths that minimize or reduce or nullify damage to living cells within the microcapsules during exposure to radiation

### Best Mode

Non-limiting examples of the invention, including the best mode, and a comparative example will be further described with reference to the accompanying drawings in which:

### BRIEF DESCRIPTION OF DRAWINGS

FIG. 1 is a schematic representation of the processes of (a) microencapsulation of rat hepatocytes and (b) surface photo-crosslinking of the microcapsules with UV-Visible light irradiation.

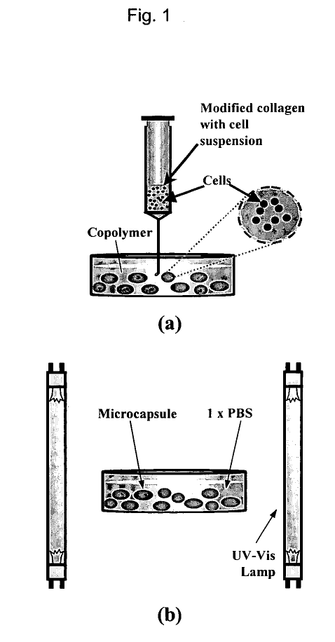

FIG. 2 shows schematic synthesis of a HEMA-MMA-MAA-MeOCPMA tetra-copolymer.

FIG. 3 shows the 400 mHz ¹H NMR spectra of (a) Polymer A in D₂O; (b) MeOCPMA in DMSO-d₆; and (c) Polymer B in D₂O.

FIG. 4 shows the changes in the UV absorption spectra of Polymer C (1.0% in PBS) after various irradiation time with UV-Visible light.

FIG. 5 shows the microcapsules being observed under an inverted light microscope. The microcapsules were formed with (a) comparative example Polymer A and (b) Polymer C (Table 1).

FIG. 6 shows the relative numbers of ruptured microcapsules prepared with comparative Polymer A and Polymers B and C as a function of vortexing time at 2000 rpm.

FIG. 7 shows the mass transfer of FITC-BSA into the microcapsules prepared from Polymer A and Polymer C with UV-Visible light irradiation.

FIG. 8 shows the effect of collagenase on degradation of collagen in microcapsules. Concentrations of collagenase used are (a) 1.5 U/mL; (b) 3 U/mL; and (c) 5 U/mL.

FIG. 9 shows the urea synthesis of hepatocytes encapsulated in microcapsules cultured in hepatozym.

FIG. 10 shows a urea synthesis of hepatocytes encapsulated in microcapsules cultured in hepatozym supplemented with 4 mM L-glutamine.

FIG. 11 shows a urea synthesis of hepatocytes encapsulated in microcapsules cultured in hepatozym supplemented with 20 ng/mL EGF and 10 nM insulin.

FIG. 12 shows a urea synthesis of hepatocytes encapsulated in microcapsules cultured in hepatozym supplemented with 20 ng/mL EGF, 10 nM insulin, and 4 mM L-glutamine.

FIG. 13 shows the relative numbers of ruptured microcapsules prepared with comparative Polymer A and polymers A, B, C′, and C as a function of vortexing time at 2000 rpm.

### MATERIALS USED IN THE EXAMPLES INCLUDE

Ethanol, 4-hydroxyacetophenone (4-HA), and 4-methoxybenzaldehyde (4-MeOBA) were purchased from Merck & Co. Inc of New Jersey, USA and Fluka Chemical Company Ltd of Buchs, Switzerland, respectively, and were used as received. Triethylamine from Fluka was purified by distillation from calcium hydride under

nitrogen. Methyl ethyl ketone supplied by J.T. Baker, a division of Mallinckrodt Baker, Inc of New Jersey, USA, was dried with anhydrous sodium sulfite prior to use. Methacryloyl chloride from Tokyo Kasei, Inc. of Japan, methyl methacrylate (MMA) and methacylic acid (MAA) from Sigma-Aldrich of St Louis, Mo., USA, and 2-hydroxyethyl methacrylate (HEMA) from Fluka were purified by vacuum distillation. 1,1′-azobis(cyclohexane carbonitrile) (ACCN) was recrystallized from ethanol. Tin (II) chloride dihydrate (SnCl₂.2H₂O) and 2-methoxyethanol were purchased from Sigma-Aldrich Biotechnology L.P. of Missouri, USA, and ninhydrin was from Merck. All other reagents were purchased from Sigma-Tau Industrie Farmaceutiche Riunite SpA of Roma, Italy unless otherwise stated.

Preparation and Characterization of Photo-Crosslinkable Anionic Copolymers

Synthesis of Anionic Copolymers

4-(4-Methoxycinnamoyl)phenyl methacrylate (MeOCPMA) was synthesized following a two-step reaction scheme as described by Reddy AVR, Subramanian K, Sainath AVS “Photosensitive polymers: Synthesis, characterization, and photocrosslinking properties of polymers with pendant alpha, beta-unsaturated ketone moiety”. J. Appl. Poly. Sci. 1998; 70(11): 2111-2120], which is incorporated herein by reference.

Copolymer of HEMA, MMA, MAA, and MeOCPMA were synthesized by solution polymerization in ethanol, using 1,1′-azobis(cyclohexane carbonitrile) (ACCN) as initiator. The initiator concentration was 0.1 mol % of the total monomers. The ratio of solvent to monomer was 10:1 (vol/wt). The polymerization was carried out with stirring at 78° C. for 4 hours in argon atmosphere. The copolymers obtained were precipitated from large excess of petroleum spirit (boiling point 40-60° C.), dried and re-precipitated in distilled water. The copolymers recovered were dissolved completely in sodium hydroxide solution and dialyzed against distilled water using dialysis tubing with molecular-weight cut-off (MWCO) of 12,000-14,000 (Spectrum Medical Industries, Houston, Tex.). The final copolymer products were freeze-dried, and characterized by using GPC, UV-Visible and ¹H NMR spectroscopy.

Molecular Weight Determination

The molecular weight (MW) and molecular weight distribution (MWD) of the copolymers were determined by gel permeation chromatography (GPC). The GPC measurements were performed on a Waters 2690 liquid chromatography system equipped with a miniDAWN™ detector and a Optilab DSP™ Interferometric Refractometer from Wyatt Technology Corporation of Santa Barbara, Calif., USA. A Polysep-GFC-P™ linear column (300×7.80 mm) from Phenomenex, Inc. of Torrance, Calif., USA and a ShodexTI Protein column (300×8 mm) of Showa Denko K.K., Tokyo, Japan connected in series were used. The eluent was a pH 7.4 buffer containing 100 mM phosphate and 150 mM sodium chloride. The measurements were performed at 37 ° C. at a flow rate of 0.50 mL/min. All copolymer samples were dissolved in the buffer and pre-filtered through a 0.22 μm Millex™-GP filter disc from Millpore Corporation of Billerica, Mass., USA, before the measurements.

Spectroscopic Measurements

Ultraviolet-visible (UV-Visible) spectra were recorded on a double monochromator UV2501PC™ spectrophotometer from Shimadzu Scientific Instruments Inc., Columbia, Md., USA, equipped with a 50W halogen lamp and a D₂ lamp. A quartz cell with 1.0 cm path length was used for the measurements.

1H-NMR spectra were recorded on a DRX400™ NMR spectrometer from Bruker BioSpin Corporation of Billercia, Mass., USA, at 400 MHz at room temperature. The sample was either dissolved with (methyl sulfoxide)-d₆ (DMSO-d₆, Aldrich) or deuterium oxide (D₂O, Merck) in a Gold label NMR tube. Chemical shifts were referred in ppm downfield from an internal standard tetramethylsilane (TMS).

Content of Photosensitive MeOCPMA Group in Copolymers

A calibration curve was obtained from the measurements of the absorption at 346 nm of a series of ethanol/water (90/10 v/v) solutions of MeOCPMA at the concentrations up to 0.025 mg/L. The copolymers were dissolved in ethanol/water (90/10 v/v), and the absorption at 346 nm was measured, and the content of the MeOCPMA group was calculated using the calibration curve.

Photoreactivity Measurements

The photoreactivity of the copolymers was evaluated by monitoring the change of UV absorption at 346 nm of a 1.0% copolymer solution in 1× PBS buffer, upon irradiation at room temperature in air in a Rayonet™ photochemical reactor equipped with 8 photochemical lamps having a maximum output at 575 nm from Southern New England Ultraviolet Co. of Branford, Conn., USA, each lamp having an output power of 30 W.

Modification of Collagen

The esterification of collagen was carried out according to the methods described by Chia S M, Leong K M, Li J, Xu X, Zeng K Y, Er P N, Gao S J, Yu H., “Hepatocyte encapsulations for enhanced cellular functions”, Tissue Eng. 2000; 6(5): 481-495 and Fraenkel-Conrat H, Olcott HS. “Esterification of proteins with alcohols of low molecular weight”. J. Biol. Chem. 1945; 161: 259-268, which are incorporated herein by reference. At the end of the reaction, the solution was dialyzed against de-ionized water at 4 ° C. using dialysis tubing with molecular-weight cut-off (MWCO) of 12,000-14,000 until the pH of external reservoir reached about 6.4, followed by freeze-drying. The modified collagen was stored at −20° C. prior to use.

Isolation of Rat Hepatocytes

Hepatocytes were isolated from male Wister rats weighing from 250 to 300 g by a two-step perfusion method described by Seglen, P O. “Preparation of isolated rat liver cells” Methods Cell Biol. 1976; 13: 29-83“, which is incorporated herein by reference. The cell viability following the final washing was estimated by the conventional Trypan Blue exclusion test described by Seglen, P O. to be 90-95%.

Preparation of Microcapsules and Photo-Crosslinking

Microencapsulation of hepatocytes was performed at room temperature. The cells was suspended at a concentration of 2.5×10⁶ cells/mL in a mixture of equal volume of medium and modified collagen (1.5 mg/mL) dissolved in 1× phosphate-buffered saline (PBS) and maintained at 4° C. before the experiment in order to prevent the gelation of the collagen solution. The hepatocyte suspension was extruded dropwise from a plastic syringe equipped with a 30.5-gauge needle into 1 wt % copolymer solution in a mixture of PBS and culture medium. A membrane was thus formed by complex coacervation of the positively charged modified collagen with the negatively charged copolymer. The microcapsules were incubated at 37° C. for 1 hour to allow the collagen to gel, and washed with PBS before further culturing in media. The microcapsules fabricated with the copolymer containing photosensitive crosslinker were irradiated for 4 minutes at room temperature in a Rayonet™ photochemical reactor equipped with 8 photochemical lamps with maximum emission wavelength of 575 nm both from Southern New England Ultraviolet Co., Branford, Conn. 06405, USA), each having an output power of 30 W.

Microcapsule Membrane Thickness

Sham capsules were prepared by extruding dropwise, the modified collagen PBS solution (1.5 mg/mL) from a plastic syringe equipped with a 30.5-gauge needle into a 1 wt % copolymer PBS solution. The microcapsules were incubated at 37° C. for 1 hour to allow the collagen to gel. The membrane thickness of the microcapsules was measured under an inverted light microscope (Olympus CK40, Tokyo, Japan).

Mechanical Stability Test

The mechanical strength of the microcapsules was evaluated by agitation of 40 sham capsules in a plastic vial together with 2 mL of PBS (1×) on a vortex mixer at about 2000 rpm. At various time intervals, the vial was removed from the mixer and the number of fractured capsules was counted. The vial was placed back on the mixer and continued for agitation. Five independent experiments were performed for each series of copolymer.

Permeability Analysis for Microcapsules

The fluorescein isothiocyanate labeled bovine serum albumin (FITC-BSA) was prepared by dissolving 50 mg of BSA in 5 mL of sodium borate buffer (pH=9.4), followed by addition of 2.5 μg of FITC. The mixture was allowed to react at room temperature for 2 hours, followed by dialysis against distilled water, and lyophilization. The diffusion of the FITC-BSA into the sham capsules was traced. The sham capsules were placed in 3 mL of FITC-BSA solution (0.5 wt %) in a cuvette. At designated time, the concentration of FITC-BSA was determined by a spectrofluorometer (FL3-11 JY Horriba Fluorolog) with excitation at 496 nm and emission at 525 nm. The tests were performed in triplicate.

Chemical Stability Of Microcapsules

Ninhydrin Reagent

Ninhydrin reagent was prepared by the method described by Allen G. et al. “Laboratory Techniques in Biochemistry and Molecular Biology: Sequencing of Proteins and Peptides”. The Netherlands: Elsevier Science 1981. p 139-141.”. 0.8 g (0.032 mol) of SnCl₂2H₂O was dissolved in 500 mL of 1× citrate buffer (pH=5.0), followed by addition of 500 mL of peroxide-free 2-methoxyethanol containing 20 g of ninhydrin. The mixture was saturated with nitrogen gas and kept at 4° C. in a dark bottle.

Ninhydrin Assay

Sham capsules prepared from 0.10 mL of modified collagen solution (1.5 mg/mL) were placed in 5.0 mL of PBS solution containing 1.5, 3.0, and 5.0 units/mL of collagenase (clostridiopeptidase A, type VII) and 0.36 mM of CaCl₂, and incubated at 37° C. At various time intervals, 0.10 mL of sample solutions were withdrawn from the vessels and replenished with fresh PBS solutions containing the same amount of collagenase. The ninhydrin assay was used to determine the amount of collagen in the microcapsules. The sample solutions were added into a tube containing 0.50 mL ninhydrin reagent. The solutions were mixed on a vortex mixer, then covered and heated in a boiling-water bath for 20 minutes. The tubes were allowed to cool down before 2.5 mL of distilled water/2-propanol (1:1 v/v) was added. The contents of the tubes were mixed thoroughly and the absorption at 570 nm of the solutions was measured within 1 hour in a quartz cell with a 1.0-cm path length.

Culturing of Encapsulated Cells

In Vitro Culture

The microcapsules were cultured in Hepatozym serum-free medium from GIBCO Laboratories of Chagrin Falls, Ohio, USA, with 10⁻⁷ M dexamethasone, 100 U/mL penicillin, and 100 μg/mL streptomycin in a 35-mm polystyrene dish in a humidified atmosphere with 5% CO₂. The effects of supplements on the functional assays were studied by addition of 10 nM insulin and 20 ng/mL epidermal growth factor (EGF) with or without 4 mM L-glutamine in the culture medium. After one day of culture, the microcapsules were incubated in the respective media with 1 mM NH₄Cl for 90 minutes before the medium was collected for urea assay. The microcapsules were then placed in the respective media again and incubated overnight.

Cellular Functions of Microencapsulated Hepatocytes

Urea-N concentrations in the media were measured calorimetrically using a commercially available test kit BUN Reagent Kit supplied by Sigma-Aldrich Co. of St Louis, Mo., USA, which was modified to fit a 96-well plate readable at 540 nm in a microplate reader. A standard calibration curve was obtained by measuring the absorption of a series of urea nitrogen standards in the concentration range of 0-45 μg/mL. Each sample (24 μL) was thoroughly mixed in a tube with 240 μL of BUN acid reagent and 160 μL of BUN color reagent, and then heated at 95° C. for exactly 10 minutes. The contents of the tubes were allowed to cool in an ice-bath for about 5 minutes, followed by spinning in a centrifuge. Finally, 100 μL of the mixture was taken for the analysis. All samples were analyzed in triplicate.

Synthesis and Characterization of Photo-Crosslinkable Anionic Tetra-Copolymers

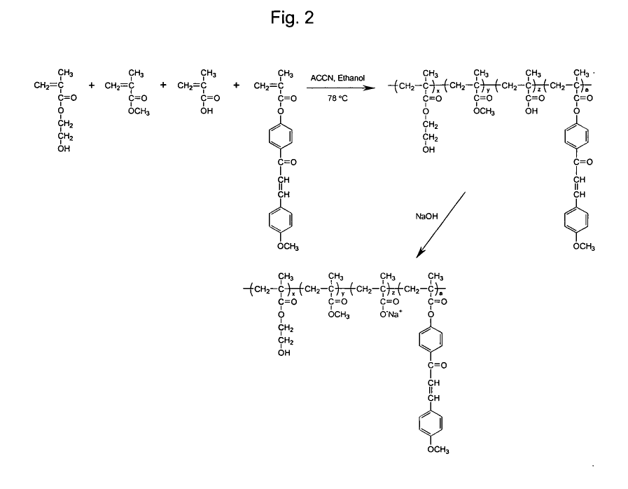

The HEMA-MMA-MAA-MeOCPMA tetra-copolymers were synthesized by free radical polymerization using 1,1′-azobis(cyclohexane carbonitrile) (ACCN) as initiator and solvent ethanol, according to the conditions shown in FIG. 2.

For comparison, a HEMA-MMA-MAA ter-copolymer without the photo-sensitive MeOCPMA was also prepared as “Polymer A”. The results of the polymerization and the molecular characteristics of the copolymers are summarized in Table 1 below.

TABLE 1
Synthesis of the HEMA-MMA-MAA-MeOCPMA tetra-copolymers and their molecular characteristics.

| | Feeding ratio (mol %) | | | | Copolymer composition (mol %) | | | | M_{w}^{c} | Mₙ^{c} | |
|---|---|---|---|---|---|---|---|---|---|---|---|
| Polymer | HEMA | MMA | MAA | MeOCPMA | HEMA^{a} | MMA^{a} | MAA^{a} | MeOCPMA^{b} | (×10⁵) | (×10⁵) | M_{w}/Mₙ^{c} |
| A | 25 | 50 | 25 | 0 | 26.2 | 45.7 | 28.1 | 0 | 2.64 | 1.86 | 1.42 |
| B | 25 | 49.5 | 25 | 0.5 | 36.6 | 42.6 | 20.6 | 0.12 | 3.38 | 1.81 | 1.86 |
| C | 25 | 47 | 25 | 3 | 29.4 | 49.0 | 20.2 | 1.39 | 2.54 | 1.49 | 1.71 |

^{a}Determined by ¹H NMR.
^{b}Determined by UV-Vis spectroscopy.
^{c}Determined by GPC.

Comparative Polymer A has no MeOCPMA monomer and therefore not cinnamoyl moiety. Polymers B and C have similar compositions and molecular weights, to comparative Polymer A except Polymers B and C contain different amounts of photo-sensitive MeOCPMA monomer that makes the copolymer photo-crosslinkable upon irradiation with UV-Visible light.

Results

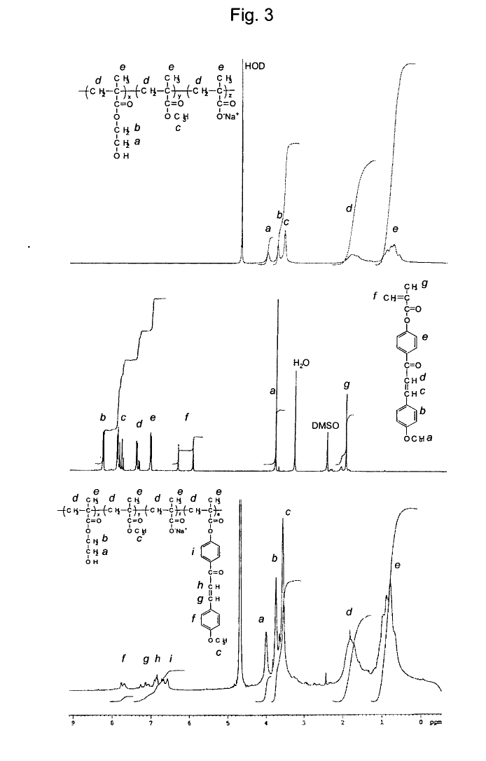

The ¹H NMR spectra of comparative polymer A, MeOCPMA, and polymer B are shown in FIG. 3. From the integration of the peaks for each proton, the composition of the two copolymers could be determined. In the spectrum of Polymer B, the multiplets at 6.5-8.5 ppm were observed, indicating the presence of the pendant cinnamoyl units. The content of the pendant cinnamoyl units was further determined by the UV absorption at 346 nm, which is much more sensitive and provides more precise data about the cinnamoyl group.

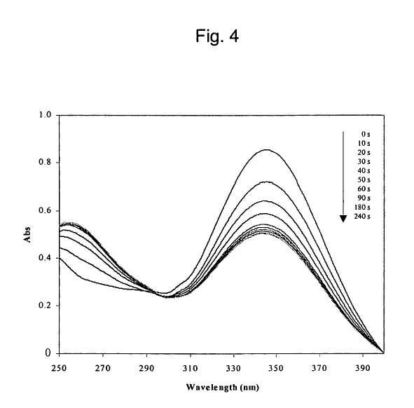

The photo-crosslinking process of Polymer C was studied by monitoring the changes in its absorption at 346 nm upon irradiation with UV-Vis light (FIG. 4) The rate of photo-crosslinking is correlated with the rate of the decrease of the absorption at 346 nm. As shown in FIG. 4, the absorption showed a steep decrease in the first 50 seconds, while further irradiation with UV-Visible light did not cause any further changes in the spectra. The result indicates that the photo-crosslinking may be achieved within a very short time period.

Preparation of Microcapsules and the Photo-Crosslinking

Microcapsules with and without hepatocytes were prepared by complex coacervation between the positively charged modified collagen and the anionic tetra-copolymers. All microcapsules were prepared with a collagen concentration of 1.5 mg/mL and a tetra-copolymer concentration of 1.0 wt %. The capsules prepared with the tetra-copolymers (Polymers B and C) were irradiated for 4 minutes with UV-Vis light using 8 photochemical lamps, to facilitate the photo-crosslinking of the outer copolymer layer. The lamps used have a maximum output emission at 575 nm because the longer wavelength light causes less, minimal or no damage to the living cells, while it's UV portion of the emission spectrum contains enough energy to trigger the photo-crosslinking of the outer layer of the microcapsules. The microcapsules without hepatocytes encapsulated, or the sham capsules, were used for the characterization in terms of their membrane thickness and permeability, mechanical strength, and chemical stability. The microcapsules with hepatocytes encapsulated were used for the cellular functional studies.

Thickness of Microcapsules

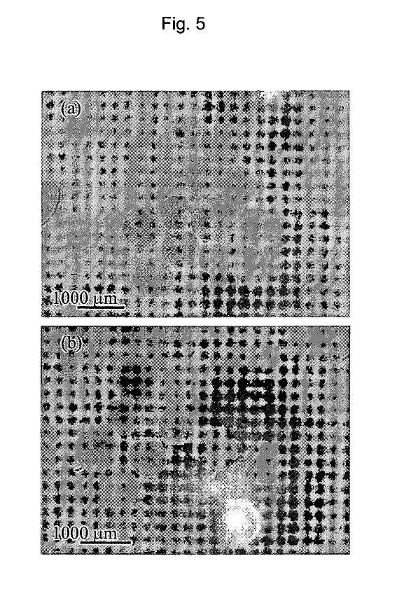

FIG. 5 shows the images of the sham capsules obtained under an inverted light microscope. The thickness of the outer copolymer layer is estimated to be ˜150 μm for capsules of about 1000 μm in diameter. The outer polymer layer that is constituted by the hydrophobic MMA and MeOCPMA, and the hydrophilic HEMA and MAA, controls the permeation selectivity of the capsules. With the use of modified collagen forming a semi-gel like inner core, a “loose” extracellular matrix configuration that mimics the in vivo situation is formed thus minimizing the impedance to mass transfer.

In embodiments where the cell encapsulation technology is applied in Bioartificial Liver Assist Devices (BLAD), the shear or abrasion effects on microcapsules will increase dramatically with diameter. Large capsules also create an internal dead volume that may impede the exchange of nutrients, oxygen, growth factors, and metabolites, leading to necrosis of the hepatocytes in the center of the microcapsules. Therefore, the size of the capsules was controlled around 900 μm.

Mechanical Stability of Microcapsules

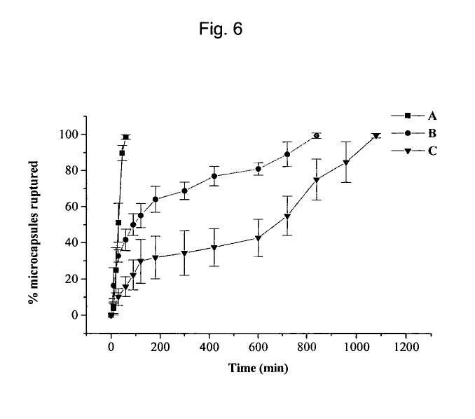

The mechanical strength of the microcapsules was evaluated by agitation of sham capsules in a plastic vial on a vortex mixer. FIG. 6 shows the relative numbers of ruptured microcapsules as a function of vortexing time. About 50% of the microcapsules formed with comparative Polymer A fractured after 30 minutes of continuous agitation, while it took about 400 and 700 minutes for 50% of microcapsules of Polymers B and C to be fractured, respectively. It can been seen that the microcapsules of Polymers B and C were significantly strengthened upon photo-crosslinking with exposure to UV-Visible light.

Among the three copolymers, Polymer C formed the strongest microcapsules, which correlates with the content of photo-crosslinkable group in the polymer chain. The results indicate that incorporation of a photo-crosslinkable group in the copolymer chain and a photo-induced crosslinking to significantly strengthen the outer membrane of the microcapsules. Microcapsules formed with Polymer C may be used in BLAD.

Permeability of Microcapsules

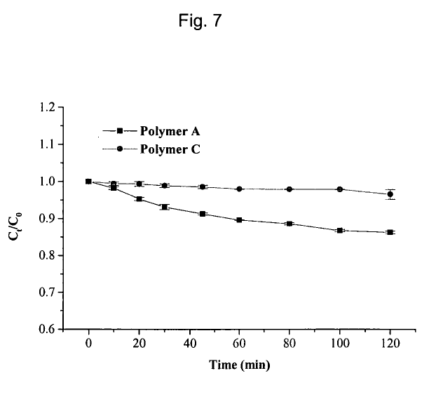

Permeability was examined by measuring the rate of transport of proteins across the capsular membrane. Previous studies have indicated that microcapsules formed with comparative Polymer A are permeable to albumin (MW ˜67,000 Da), which is one of the secreted proteins of hepatocytes, and proteins smaller than albumin, but impermeable to immunoglobulins (MW ˜150,000 Da) or larger molecules of the immune system. The concentration of FITC-BSA in the extracapsular solution was monitored, which decreased when FITC-BSA diffused into the microcapsules. For comparison, the concentration of the protein was normalized and expressed as Cₜ/C₀, where Cₜ and C₀ are the concentrations of FITC-BSA remained in the extracapsular solution at time t and time 0, respectively. FIG. 7 shows the Cₜ/C₀ changes as a function of time. The diffusion of FITC-BSA into the microcapsules formed with Polymer C was slower than that with Polymer A, indicating that the membrane formed with the photo-crosslinkable capsules had smaller pore size providing better immunoprotection of the encapsulated cells.

Chemical Stability of Microcapsules

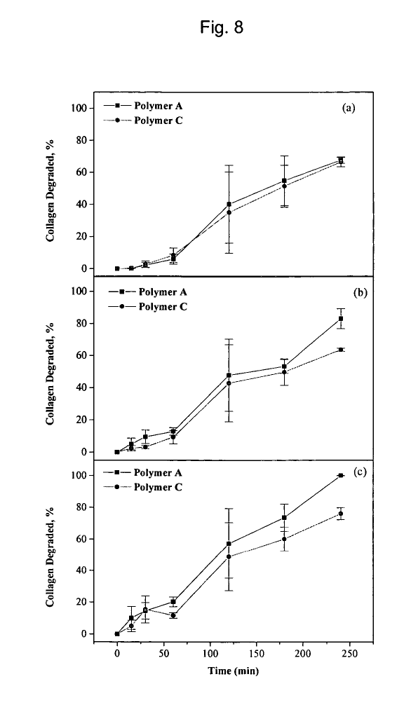

The effect of collagenase on the enzymatic degradation of the microcapsules formed with the copolymers is shown in FIG. 8. The microcapsules formed with Polymer C were photo-crosslinked prior to putting into the collagenase solution. The photo-crosslinking took place on the surface of the microcapsules and not the inner core of the microcapsules. In the presence of the collagenase, the enzyme diffused inwards from the surface to degrade the inner collagen core. This caused the capsule size to reduce, followed by total collapse of the microcapsules. As shown in FIG. 8, the collagen degradation rate was generally higher when a higher collagenase concentration was used. The degradation of collagen in microcapsules formed with Polymer C was found to be slower than that with comparative Polymer A, and the difference is more significant at higher collagenase concentrations. These results indicate that the photo-crosslinking of capsular membrane enhanced the chemical stability of the microcapsules.

Cellular Functions of Encapsulated Hepatocytes

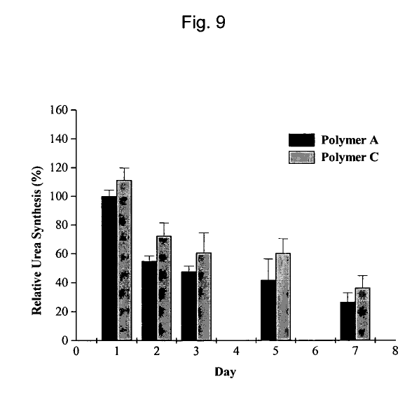

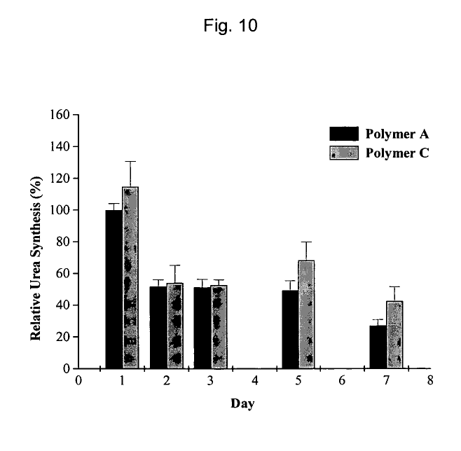

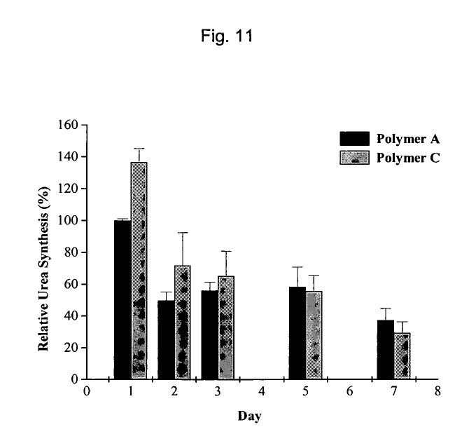

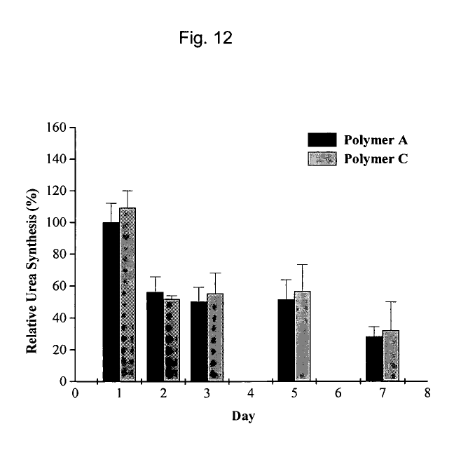

Urea synthesis is one of the most important indicators of the hepatocyte functions. FIGS. 9, 10, 11, and 12 show the relative levels of urea synthesized by hepatocytes encapsulated in microcapsules formed with Polymers A and C, and cultured in hepatozym, hepatozym with L-glutamine, hepatozym with EGF and insulin, hepatozym supplemented with L-glutamine, EGF, and insulin, respectively. In general, the varying supplements added to the culture medium did not trigger enhancement to urea synthesis. However, microcapsules formed with Polymer C exhibit higher levels of urea synthesis than those with comparative Polymer A. Without being bound by theory it is thought that this may indicate that the photo-crosslinking of the microcapsules enhanced the cellular functions of the encapsulated hepatocytes by providing a more stable environment for the hepatocyte culture.

Optimal composition of MeOCPMA

To determine the optimal composition of the photo-crosslinkable monomer unit (MeOCPMA) in a tetra-copolymer, more copolymers were synthesized with different feeding ratios. When the feeding ratio of MeOCPMA was higher than 4 mol %, the resulted copolymers were nearly not water soluble, which are practically not useful in the encapsulation. For those with feeding ratio of MeOCPMA less than 3, the copolymers synthesized are listed in Table 2 below.

TABLE 2
Synthesis of the HEMA-MMA-MAA-MeOCPMA tetra-copolymers
and their molecular characteristics.

| Poly- | Copolymer Composition (mol %) | | | | M_{w} | Mₙ | M_{w}/ |
|---|---|---|---|---|---|---|---|
| mer | MMA | HEMA | MAA | MeOCPMA | (×10⁵) | (×10⁵) | Mₙ |
| A | 45.7 | 26.2 | 28.1 | 0 | 2.64 | 1.86 | 1.42 |
| B | 42.6 | 36.6 | 20.6 | 0.12 | 3.38 | 1.81 | 1.86 |
| C′ | 49.9 | 43.8 | 5.4 | 0.93 | 1.16 | 0.83 | 1.40 |
| C | 49.0 | 29.4 | 20.2 | 1.39 | 2.54 | 1.49 | 1.71 |

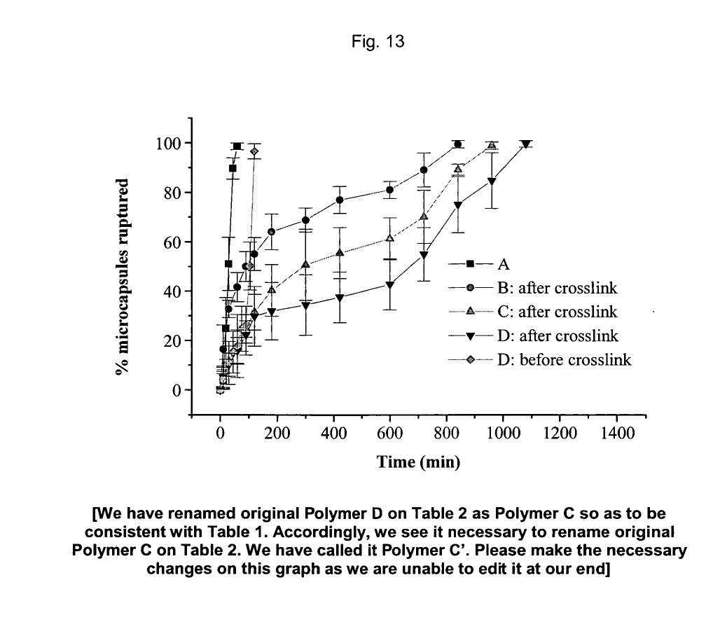

Results of mechanical stability studies of microcapsules formed with Polymers A, B, C′, and C are shown in FIG. 13. It can be seen that after photo-crosslinking, the mechanical stability of all microcapsules with MeOCPMA significantly increased. Polymer C with 1.39 mol % MeOCPMA showed the best mechanical stability.

For microcapsules with copolymer C, a comparison of mechanical stability before and after the photo-crosslinking was carried out. Before crosslinking, the microcapsules showed very low mechanical stability, which is similar to that of microcapsules formed with copolymer A (without MeOCPMA). In contrast, the mechanical stability of the microcapsules formed with copolymer C significantly increased after the photo-crosslinking.

### Applications

Upon irradiation with UV-Visible light, the photo-sensitive double bond of the monomer MeOCPMA undergoes a [2+2]-cycloaddition reaction with the other MMA-HEMA-MAA monomers. It is thought that the strong covalent bonding within the outer polymer layer provides the microcapsules with good mechanical strength and good chemical stability.

The cellular function of the living cells and the function of the bioactive substance encapsulated therein is enhanced, possibly by providing a more stable environment for the materials through the improved mechanical and chemical properties of the microcapsules.

Accordingly, the microcapsules are very useful and a plurality of the microcapsules can be applied to a wide range of applications including general living cell microencapsulation for maintaining cell functions, hepaocyte microencapsulation for bioartificial liver assist devices (BALD), as cell scaffolding materials in stem cell technology, for cell delivery and cell therapy in tissue engineering, and encapsulation of therapeutic agents or other bioactive substances for their controlled delivery.

The cross-linking of the MeOCPMA monomer takes places under UV-Visible light. As UV-Visible light is at a longer wavelength of light, it is relatively safer to work with in industrial infrastructure environments. The present invention therefore also provides a simplified and safe method for producing encapsulated microcapsules, which may result in cost and time savings for the industrial scale production of the microcapsules.

The longer wavelength UV-Visible light also causes minimal or no damage to the living cells or bioactive substances encapsulated in the microcapsules. This is advantageous as the cellular function of the bioactive substances will not be compromised during the manufacturing process.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A microcapsule comprising:
a permeable polymer membrane comprising a plurality of cinnamoyl groups, the cinnamoyl groups being capable of forming cross-links upon exposure to radiation; and
a bioactive substance encapsulated by the permeable membrane.

2. A microcapsule as claimed in claim 1, wherein a monomer group has the cinnamoyl groups incorporated into a polymer backbone of the polymer membrane.

3. A microcapsule as claimed in claim 1, wherein the permeable polymer membrane comprises:
an inner polymer layer comprising a first polymer; and
an outer polymer layer comprising a second polymer.

4. A microcapsule as claimed in claim 3, wherein the first polymer has a first electrical charge and the second polymer has a second electrical charge opposite to the first electrical charge.

5. A microcapsule as claimed in claim 3, wherein the outer polymer layer comprises a copolymer that comprises the plurality of cinnamoyl groups.

6. A microcapsule as claimed in claim 3, wherein the permeable polymer membrane is selectively permeable and the outer polymer layer comprises at least one hydrophobic group and at least one hydrophilic group for controlling the selectivity of the membrane.

7. A microcapsule as claimed in claim 6, wherein the at least one hydrophobic group of the outer polymer layer is adjacent to the inner polymer layer and the outer polymer at least partially surrounds the inner polymer layer.

8. A microcapsule as claimed in claim 3, wherein the second polymer is an anionic tetra-copolymer comprising groups derived from hydroxyethyl methacrylate, methyl methacrylate, methacrylic acid and methoxycinnamoyl phenyl methacrylate.

9. A microcapsule as claimed in claim 3, wherein the first polymer is a cationic biopolymer comprising collagen modified to have a pKI of at least about 9.

10. A microcapsule as claimed in claim 1, wherein the bioactive substance is suspended within a cationic biopolymer comprising collagen modified to have a pKI of at least about 9.

11. A microcapsule as claimed in claim 1, wherein the bioactive substance comprises cells and the permeable polymer membrane is a selectively permeable membrane that is permeable to one or more materials necessary to sustain the normal metabolic functions of cells and to products released by the cells and impermeable to one or more immune system components associated with immune rejection in an animal.

12. A microcapsule as claimed in claim 1, wherein the bioactive substance comprises living cells or genetically engineered cells selected from the group consisting of: hepatocyte cells, hematopoietic cells, epithelial cells, secretory cells, ciliated cells, contractile cells, sensory cells and neuronal cells and one or more combinations thereof.

13. A microcapsule as claimed in claim 1, wherein the bioactive substance is selected from the group consisting of: therapeutic compounds, neurological compounds, vitamins, vitamin derivatives, growth factors, glucocorticosteroids, steroids, antibiotics, anti-bacterial compounds comprising bacteriocidal and bacteriostatic compounds, anti-viral compounds, anti-fungal compounds, anti-parasitic compounds, tumoricidal compounds, tumoristatic compounds, toxins, enzymes, enzyme inhibitor proteins, peptides, minerals, neurotransmitters, lipoproteins, glycoproteins, immunomodulators, immunoglobulins and corresponding fragments, dyes, radiolabels, radiopaque compounds, fluorescent compounds, fatty acid derivatives, polysaccharides, cell receptor binding molecules, anti-inflammatory compounds, anti-glaucomic compounds, mydriatic compounds, anesthetic compounds, nucleic acids, polynucleotides and combinations of one ore more thereof.

14. A microcapsule as claimed in claim 1, wherein the molar concentration of cinnamoyl groups provided in the permeable polymer membrane is in the range between about 0.01 mol % to about 4 mol %.

15. A microcapsule as claimed in claim 1, wherein the outside diameter of the microcapsule is between about 500μm to about 1,500μm.

16. A microcapsule as claimed in claim 1, wherein the cinnamoyl groups are derived from a monomer selected from the group consisting of: 4-(4-Methoxycinnamoyl)phenyl methacrylate; 3,4-dimethoxycinnamoyloxyethyl methacrylate, 3,4,5-trimethoxycinnamoyloxyethyl methacrylate, cinnamoyloxyethyl methacrylate and one or more combinations thereof.

17. A microcapsule comprising:
a permeable polymer membrane comprising at least one crosslinking group derived from at least two cinnamoyl groups; and
a bioactive substance encapsulated by the permeable polymer membrane.

18. A microcapsule comprising:
a permeable polymer membrane comprising:
an inner polymer layer comprising a first polymer having a first electrical charge;
an outer polymer layer comprising a second polymer having a second electrical charge opposite to the first electrical charge, the second electrical charge being sufficient to form a complex with the first polymer of the inner polymer layer;
a plurality of monomer groups comprising cinnamoyl groups incorporated into a backbone of the first polymer or the second polymer, the cinnamoyl groups being capable of forming cross-links when exposed to light at a wavelength in the range from 340 nm to 700 nm; and
a bioactive substance encapsulated by the permeable membrane.

19. A method of preparing a microcapsule, comprising the steps of:
(a) providing a first solution of a first polymer;
(b) providing a second solution of a second polymer;
(c) providing a bioactive substance in the first or the second polymers; and
(d) introducing the first solution, the second solution and the bioactive substance to form a permeable polymer membrane at least partially surrounding the bioactive substance;
wherein at least one of the first polymer and the second polymer has a plurality of cinnamoyl groups capable of forming cross-links upon exposure to radiation.

20. A method according to claim 19 comprising the additional step of exposing the permeable polymer membrane to radiation so that the cinnamoyl groups form cross-links within the permeable polymer membrane.

21. A pharmaceutical composition comprising a pharmacologically effective plurality of the microcapsules of claim 1, together with a pharmacologically acceptable carrier.

22. Use of one or more microcapsules as claimed in claim 1, in a liver assist device.

23. Use of one or more microcapsules as claimed in claim 1, as a stem cell scaffold material.

24. Use of one or more microcapsules as claimed in claim 1, in the controlled delivery of therapeutic agents.
